(19) United States
(12) Patent Application Publication
(10) Pub. No.: US 2026/0081022 A1
(43) Pub. Date: Mar. 19, 2026

(54) Methods for Performing Analyses of Biological Samples and Computer Program Product

(71) Applicant: Robert Bosch GmbH, Stuttgart (DE)

(72) Inventors: Ramona Hartmann, Goeppingen (DE); Anna-Lena Schrof, Waiblingen (DE); Marc Koltermann, Ludwigsburg (DE)

(21) Appl. No.: 19/323,685

(22) Filed: Sep. 9, 2025

(30) Foreign Application Priority Data
Sep. 10, 2024 (DE) 10 2024 208 575.8
Aug. 28, 2025 (DE) 10 2025 134 426.4

(51) Int. Cl. G16H 50/20; G16H 10/40

(52) U.S. Cl. CPC G16H 50/20; G16H 10/40

(57) ABSTRACT

A method for performing analyses of biological samples using at least one diagnostic device, in particular in a diagnostic system, includes (i) checking whether an internal value stored in a software module in a control device of the diagnostic device corresponds to a zero value in analyses when a biological sample is or was fed to the diagnostic device by way of a consumable material specific to the diagnostic device, (ii) analyzing the biological sample by the diagnostic device if the internal value deviates from the zero value, and (iii) changing the internal value in the software module of the diagnostic device by an operation value of an analysis.

This application claims priority under 35 U.S.C. § 119 to (i) application no. DE 10 2024 208 575.8, filed on Sep. 10, 2024 in Germany, and (ii) application no. DE 10 2025 134 426.4, filed on Aug. 28, 2025 in Germany, the disclosures of which are incorporated herein by reference in their entirety.

The disclosure relates to a method for performing analyses of biological samples using at least one diagnostic device. The disclosure also relates to a computer program product which is designed to carry out a method according to the disclosure.

### BACKGROUND

The applicant's Vivalytic® brand is known as a universal platform for molecular diagnostics, in which various types of patient samples (e.g., oral swabs) are tested in a fully automated manner. For this purpose, the diagnostic device performs sample preparation, analysis, and sample evaluation. During analysis, the patient sample is placed in a test cartridge designed as consumable material specific to the diagnosis, which is inserted into the diagnostic device. The diagnostic device then performs a fully automated test of the biological sample, for example using a PCR procedure. When performing the analysis, it is also necessary for the diagnostic device to control appropriate diagnostic software. This requires the diagnostic device to perform capturing of the relevant data from the test cartridge, which is done using a barcode scanner in the above example. Furthermore, the diagnostic device known to date can also be connected via an Internet connection to a web-based control unit (Vivasuite®), which is used to enable the management, organization, and updating of data in the diagnostic device in question.

Another diagnostic device of the applicant is known under the name Vivatmo. In the diagnostic device according to Vivatmo, breathing air is fed to the diagnostic device via a replaceable mouthpiece designed as a consumable material. The diagnostic device determines the nitrogen monoxide concentration of the breath sample in order to diagnose respiratory tract inflammation, for example.

### SUMMARY

The method according to the disclosure for performing out analyses of biological samples and the computer program product have the advantage that the flexibility of the diagnostic system is improved.

The disclosure's advantages are particularly evident in the operation of networked diagnostic systems comprising a plurality of diagnostic devices. This enables dynamic control of the analysis credits available in the system. This leads to a significant improvement in the overall technical operational readiness and throughput of the diagnostic system. In particular, continuous operation is promoted even when the individual diagnostic devices are not being used equally, as analysis credits can be flexibly displaced between the devices and a central pool. The disclosure thus relates to the targeted control of the internal functioning of the technical system in order to optimize its operating status and minimize downtime.

The technical basis for this is a system architecture with a device-specific internal value on the diagnostic device and a central availability value on an external control unit. The operations defined in the disclosure, which are executed by the system's processors, serve to technically implement this system control. They enable a subset-based redistribution of analysis credits by specifically and oppositely changing the internal value and the availability value for optimized functionality. The disclosure thus enables efficient allocation of the digital resource “analysis credits”within the technical system.

Advantageous embodiments of the method and computer program product according to the disclosure are the subject of the following description and figure description.

The features described and claimed in the method shall also be considered to be disclosed in terms of the device.

The method for performing analyses of biological samples using at least one diagnostic device, in particular in a diagnostic system, comprises the following method steps:
checking whether an internal analysis value stored in a software module in a control device of the diagnostic device corresponds to a zero value when a biological sample is or was fed to the diagnostic device using a consumable material specific to the diagnostic device.
analyzing the biological sample by the diagnostic device if the internal value deviates from the zero value, in particular if it is greater than the zero value.
changing the internal value in the software module of the diagnostic device by an operation value of an analysis if the analysis of the biological sample is or has been performed.

According to the disclosure, the following method steps are provided:
performing an initial operation with an existing communication connection with bi-directional data exchange between the diagnostic device and application software of an external control unit, whereby the internal value is changed, in particular reduced, depending on a first variable value representative of a number of analyses, and an availability value stored in the application software of the external control unit is changed, in particular increased, depending on the first variable value, and/or
performing a second operation with an existing communication connection with bi-directional data exchange between the diagnostic device and one/the application software of one/the external control unit, whereby one/the availability value stored in the application software of the external control unit is changed, in particular reduced, depending on a second variable value representative of a number of analyses, in particular reduced, and the internal value is changed, in particular increased, depending on the second variable value.

The method according to the disclosure for performing analyses of biological samples offers the advantage that a number of analyses that can still be performed with the diagnostic device can be determined dynamically, thereby specifically controlling the operational readiness of the diagnostic device or a more comprehensive diagnostic system. It may be advantageous to provide that this functionality can be activated and deactivated for a diagnostic device, in particular temporarily, i.e., that before performing an analysis, a check is made as to whether a software module is activated or deactivated in a control device of the diagnostic device, wherein, if the software module is deactivated, the sample is analyzed, and if the software module is activated, a check is performed to determine whether an internal analysis value stored in the software module of the diagnostic device corresponds to the zero value and/or the blocking value. Thus, the disclosure can advantageously define an operating time of the diagnostic device for a predetermined and preferably dynamic number of analyses, which supports, for example, an interruption for a functional test or maintenance of the diagnostic device before a release for further operation.

In addition, the method can be used advantageously to check whether approvals or rights of use have been obtained and/or acquired for the consumable materials used, such as test cartridges. This has the advantage that the use of consumable materials that may not be certified or may be unsuitable in terms of quality requirements, for example, can at least be restricted and/or prevented. The rights of use for a corresponding number of consumable materials must be acquired, for example, via a distribution channel approved by the operator of an external application software program, and can be activated via the external application software program after establishing a communication connection with the diagnostic device, in the broadest sense as analysis credits for the diagnostic device. The analysis credit, which represents in particular a number of analyses available on the device, is represented by the respective internal value in the software module of the diagnostic device. In addition to the analysis credit, the availability value stored in an external control unit and/or application software defines a centrally stored and available availability credit. The availability value and thus the disposable credit balance can also be changed, in particular increased, by registering new consumable material, wherein it is also possible to transfer analysis credits back to the disposable credit balance from a diagnostic device. The reversal of analysis credits to the disposable credit balance can involve either a complete reversal or the reversal of a partial amount of the analysis credit balance. The first variable value defines the amount and/or partial amount that is deducted from the analysis credit of a diagnostic device and credited to the disposable credit. The first variable value thus corresponds in the broadest sense to a reversal quantity.

The alternative or additional option of transferring analysis credits from the disposable credit to the diagnostic device also prevents the diagnostic device from being blocked as long as analysis credits and/or disposable credits are still available in the diagnostic system. In this case, the analysis credit of the diagnostic device can be increased by transferring available credit, wherein the amount deducted from the available credit and by which the analysis credit is increased is defined by the second variable value, which represents in particular a number of analyses to be transferred. The second variable value therefore corresponds in the broadest sense to a top-up quantity.

The zero value and/or lock value can be any value selected, wherein the number zero can be provided in particular. The operation value of an analysis can also be any value, wherein the number one can be provided in particular. The first and second operations can be any operations, in particular those performed by a processor of the control device or the external control unit, which lead to a change in the internal value and/or the availability value, wherein addition and subtraction of natural numbers can preferably be provided. Other implementations, such as multiplication of binary numbers, are also conceivable. Particularly preferably, the first and second operations each comprise two inverse operations, for example a reduction of the internal value and an increase of the availability value, or vice versa, by the first or second variable value, respectively. In other words, the available credit balance and analysis credit balance, as well as their reversal and replenishment, and all other values comprised in the method, can be represented in any number range and/or value and changed using any operation, which makes it possible to assign the value and/or operation to the presence or absence of credit and/or a directed change in credit. Thus, for example, an increase in the analysis credit does not necessarily mean an increase in the internal value, but merely a change in the internal value which is suitable for being implemented by the application software and/or the control device and/or the software module as an increase in the analysis credit.

The method according to the disclosure basically enables both autonomous operation of the diagnostic device without a communication connection to external application software (a so-called “offline mode”) and operation with an existing communication connection (a so-called “online mode”). In both operating modes, the internal value of analyses must not be zero in order to perform an analysis of the biological sample.

The biological sample may in particular be a human or animal sample such as sputum, blood, urine, exhaled air, a swab or a tissue sample. In particular, the analysis may involve the detection of an analyte or pathogen in the sample. A consumable material can be understood in particular as a disposable item, as a consumable material intended for single use only. In alternative embodiments, consumable material can also be understood as material that can be used several times, for example after cleaning and/or after replacing a part of the consumable material.

The method is particularly preferred when performed on at least two diagnostic devices in a diagnostic system. This has the advantage that, in the event of uneven use of several available diagnostic devices, the analysis credit of a first diagnostic device does not remain unused, while a more frequently used at least second diagnostic device is blocked due to insufficient analysis credit. This makes it possible to redistribute and/or distribute analysis credits booked on one diagnostic device, as well as available credits, to another diagnostic device. In order to achieve a redistribution of analysis credits from one diagnostic device to at least a second diagnostic device, a reversal amount can be transferred from the analysis credits of the first diagnostic device to the available credits, and a top-up amount can be transferred from the available credits to the analysis credits of at least the second diagnostic device.

According to an advantageous embodiment, it may be provided that the method for performing analyses of biological samples is carried out using at least two diagnostic devices, in particular in a diagnostic system, wherein a biological sample is fed to a diagnostic device using a specific to the diagnostic device consumable material and is analyzed by the diagnostic device, wherein, before performing the analysis of the sample, it is checked whether an internal analysis value stored in a software module in a control device of the diagnostic device corresponds to a zero value and/or a blocking value, wherein, in the case of an internal value that deviates from the zero value and/or blocking value, the analysis of the sample is performed, wherein, in the case of an internal value that corresponds to the zero value and/or blocking value, no analysis of the sample is performed, and wherein, after each analysis by the diagnostic device, the internal value in the software module of the diagnostic device is changed by an operation value of an analysis, wherein, if there is a communication connection with bi-directional data exchange between the diagnostic devices and application software of an external control unit, a first operation is performed, whereby the internal analysis value is changed, in particular reduced, depending on a first variable value of analyses, and an availability value of analyses stored in the application software/external control unit is changed, in particular increased, depending on the first variable value, and/or a second operation is performed, whereby the availability value is changed, in particular reduced, in dependence on a second variable value of analyses, and the internal analysis value is changed, in particular increased, in dependence on the second variable value.

According to a further advantageous embodiment, it may be provided that if the first and/or second operation would change the internal value and/or the availability value to a value which, starting from the original internal value and/or availability value, lies beyond the zero value, in particular falls below or exceeds the zero value, the first and/or second operation is not executed and/or is aborted. This is a technical protective measure to maintain the integrity of credit assets in the system and prevent a faulty system state. An error message displayed via a user interface of the application software is particularly advantageous if the first or second operation is canceled or not executed.

According to an advantageous embodiment, it may be provided that the application software checks whether a user input has taken place at a user interface of the external control unit and sets the first and/or second variable value according to the user input. This makes it possible to enter the variable value manually, which is advantageous. It is particularly advantageous to use user input to determine from which of a preferred plurality of diagnostic devices analysis credits are deducted via a reversal amount and to which diagnostic device additional analysis credits are credited. Furthermore, it may also be advantageous to provide that analysis credits from a diagnostic device are deducted and credited to the availability value without being immediately redistributed.

According to an advantageous embodiment, it may be provided that the application software calculates a proportion of equal size for a plurality of diagnostic devices from the availability value, in particular automatically when the availability value reaches a value deviating from the zero value, and determines the second variable value according to the proportion. In other words, available credits are distributed equally across the analysis credits of the diagnostic devices comprised in the diagnostic system via the top-up amount. This has the advantage of ensuring that the diagnostic devices in the diagnostic system have the same number of analyses available. The distribution of equal proportions can preferably be done automatically if the availability value is changed to a value other than zero via the registration of new consumable materials or a change, in particular an increase, in the availability value by the first operation. The proportions are whole numbers, wherein it may be stipulated that the availability value is not distributed in full if this would result in proportions that are not whole numbers.

According to an advantageous embodiment, it may be provided that usage data relating to the analyses performed, in particular by a plurality of diagnostic devices, preferably at least the number of analyses performed, is transmitted to the application software, wherein the application software stores the usage data and, on the basis of the usage data, creates and stores an individual usage profile for each of the diagnostic devices based on the usage data. This provides the advantage of obtaining at least data on the usage frequency of a diagnostic device, wherein further data, such as the types of analyses performed and/or the average number of analyses performed over a defined period of time, are preferably recorded and used to create usage profiles.

According to an advantageous embodiment, it may be provided that the application software calculates individual proportions for a plurality of diagnostic devices from the availability value, in particular automatically when the availability value reaches a value deviating from zero, on the basis of the usage profiles, and determines the second variable value for the diagnostic devices according to the respective corresponding individual proportion. This allows the allocation of the assigned additional analysis credit in the form of the proportionate second variable value and/or additional quantity to be optimized in relation to the usage frequency of a diagnostic device by advantageously adjusting the assigned analysis credit to the individual usage intensity of the diagnostic device. It may be advantageous to take other data contained in the usage profiles into account when calculating the individual proportions, such as the type of analyses performed on a diagnostic device.

According to an advantageous embodiment, it may be provided that the application software creates an individual usage forecast for a plurality of diagnostic devices based on the usage profiles, wherein the difference between an expected usage frequency, in particular for 14 days from the creation of the usage forecast, and the internal value is calculated. In other words, the application software generates a forecast as to whether the analysis credit of the corresponding diagnostic device is sufficient to operate the diagnostic device over the defined period, preferably 14 days, without the internal value and/or the analysis credit reaching zero and the diagnostic device being blocked.

According to an advantageous embodiment, it may be provided that a plurality of diagnostic devices are assigned on the basis of the usage forecast to a category, in particular to a plurality of categories, which is highlighted in a graphical user interface of the application software, in particular optically, particularly preferably as a color-coded traffic light. At least three categories are particularly advantageous, namely a first category for diagnostic devices whose analysis credit is sufficient for two weeks of operation according to the usage forecast, a second category for diagnostic devices whose analysis credit is sufficient for less than two weeks of operation according to the usage forecast, and a third category for diagnostic devices whose internal value and/or analysis credit is zero. A user interface preferably displays graphically which category a diagnostic device is assigned to, in particular with a green color code for the first category described above, a yellow color code for the second category, and a red color code for the third category.

According to an advantageous embodiment, it may be provided that, when assigning a diagnostic device to a category, the application software determines a first and/or second variable value according to a difference between the internal value and a threshold value calculated on the basis of the usage forecast for assignment to another category. This makes it possible, for example, for a diagnostic device whose analysis credit according to the usage forecast is not sufficient for further operation for more than two weeks, to calculate by what value the internal value must be increased or multiplied or otherwise changed in order to be assigned to a category of diagnostic devices whose analysis credit according to the usage forecast is sufficient to enable operation for at least two weeks.

According to a particularly preferred embodiment, it may be provided that the method for performing analyses of biological samples using at least one diagnostic device, in particular in a diagnostic system, is carried out such that a biological sample is fed to the diagnostic device using specific to the diagnostic device consumable material and analyzed by the diagnostic device, wherein, before performing the analysis of the sample, it is checked whether an internal analysis value stored in a software module in a control device of the diagnostic device is greater than zero, wherein, if the internal analysis value is greater than zero, the analysis of the sample is performed, wherein if the internal value is zero, the sample is not analyzed, and wherein after each analysis by the diagnostic device, the internal value in the software module of the diagnostic device is reduced by the value one, wherein, if there is a communication connection with bi-directional data exchange between the diagnostic device and application software of an external control unit, a first variable value of analyses is subtracted from the internal analysis value and a value of analyses stored in the application software/external control unit is increased by the first variable value of analyses and/or that, when a second variable value of analyses is subtracted from the availability value, the internal analysis value is increased by the value of the second variable value. Advantageously, the internal value and the first and second variable values in this embodiment correspond to a number based on natural numbers and zero, and can thus also be referred to as an internal number and first and second variable numbers.

According to an advantageous embodiment, it may be provided that if the first variable value is greater than the internal value and/or the second variable value is greater than the availability value, no reduction of the internal value and/or the availability value takes place. In other words, this prevents the internal value and the availability value from reaching negative values. An error message is displayed via a user interface of the application software, which is particularly advantageous if the specified first or second variable value exceeds the internal value or availability value.

The disclosure also comprises a control device for a diagnostic device for performing analyses of biological samples, in particular in a diagnostic system, which is configured to perform the following method steps:
checking whether an internal analysis value stored in a software module of the control device corresponds to a zero value when a biological sample is or was fed into the diagnostic device using a consumable material specific to the diagnostic device.
approval of an analysis of the biological sample by the diagnostic device if the internal value deviates from the zero value, in particular if it is greater than the zero value. changing the internal value in the software module of the diagnostic device by an operation value of an analysis if the analysis of the biological sample is or has been performed, and
performing an initial operation with an existing communication connection with bi-directional data exchange between the control device and application software of an external control unit, whereby the internal value is changed, in particular reduced, depending on a first variable value representative of a number of analyses, and an availability value stored in the application software of the external control unit is changed, in particular increased, depending on the first variable value, and/or
performing a second operation with an existing communication connection with bi-directional data exchange between the control device and one/the application software of one/the external control unit, whereby one/the availability value stored in the application software of the external control unit is changed, in particular reduced, depending on a second variable value representative of a number of analyses, in particular reduced, and the internal value is changed, in particular increased, depending on the second variable value.

The disclosure also comprises a diagnostic device for performing analyses of biological samples, in particular in a diagnostic system with a control device of the type described above.

The disclosure also comprises a computer program product comprising instructions which, when executed by a computer, cause the computer to perform a method for performing analyses of biological samples using at least one diagnostic device, in particular in a diagnostic system, according to one of the preceding embodiments.

### BRIEF DESCRIPTION OF THE DRAWINGS

FIG. 1 Schematic overview of the method used in a diagnostic system

FIG. 2 Schematic overview of the method for classifying diagnostic devices into categories

### DETAILED DESCRIPTION

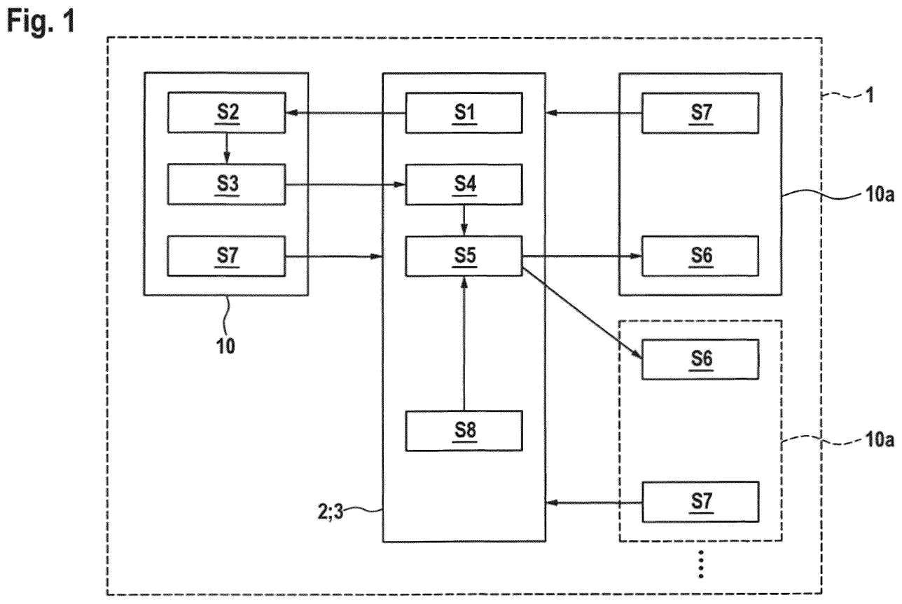

FIG. 1 shows a schematic overview of a possible procedure in a diagnostic system 1 comprising at least two diagnostic devices 10; 10a. An embodiment with only one diagnostic device 10 or a larger plurality of diagnostic devices 10; 10a is also possible. In a first method step S1, a first variable value N1 is determined. The first variable value N1 is a measure of a reversal amount, which in particular represents a number of analyses to be transferred, which can be transferred and/or reversed from an analysis credit of a diagnostic device 10; 10a, which is represented by the internal value A1; A1a, to the availability value AN, as a central availability credit. This can be done via user input at a user interface 4, at the external control unit and/or application software 2, wherein the application software 2 checks whether such user input has taken place and sets the first variable value N1 accordingly. The first variable value N1 can also be set automatically by the application software 2, for example, if the internal value A1; A1a of a diagnostic device 10; 10a reaches the zero value ZV and a blocking of the diagnostic device 10; 10a is to be automatically prevented by reversing and replenishing analysis credits, for example, in accordance with an instruction that can be set in the application software 2.

In a second method step S2, the variable value ZV is transmitted to a diagnostic device 10, in particular via a bi-directional data connection. In a method step S3, the diagnostic device 10, in particular the software module 12, first checks whether executing the first operation with the first variable value N1 would change the internal value A1 to a value that is beyond the zero value ZV based on the original internal value A1, wherein if such a change would occur, the first operation is not executed and, in particular, an error message is displayed, advantageously in the application software 2. If the first variable value N1 has a value that does not cause the zero value ZV to be exceeded, the internal value A1 is changed depending on the first variable value N1 by executing and/or starting the first operation, for example, subtracting the first variable value N1 from the internal value A1, and the new internal value A1 is stored.

In a method step S4, a confirmation is sent from the diagnostic device 10 to the application software 2 that the change to the internal value A1 has been carried out, depending on the first variable value N1. The changed internal value A1 is stored by the application software 2 and the availability value AN is changed depending on the first variable value N1 by continuing the first operation, for example by adding the first variable value N1 to the availability value AN. This results in a reversal of the analysis credit of diagnostic device 10 to the availability value AN, and the first operation is completed. The first operation and the associated change in the internal value and availability value may comprise one or more arbitrary operations in any number or value range, for example, addition of natural numbers, which may enable the functionality of a reversal in the broadest sense. Advantageously, the first operation comprises two inverse operations, such as subtracting the first variable value N1 from the internal value A1, A1a and adding the first variable value N2 to the availability value AN.

The availability value AN can be changed in addition to or as an alternative to method steps S1 to S4, in method step S8, by registering new consumable materials and/or activating new analysis credits. The availability value AN thus represents a central availability credit and/or a credit pool that is not assigned to any diagnostic device 10; 10a.

The second variable value N2 is determined in a method step S5 and represents a replenishment quantity, which in particular also represents a number of analyses to be transferred, which can be deducted from the availability value AN and/or the disposable credit and credited to an analysis credit of a diagnostic device 10; 10a by changing, in particular increasing, the internal value A1; A1a. This can be done by a user input at a user interface 4, wherein the application software 2 checks whether such a user input has taken place and sets the second variable value N2 accordingly. The second variable value N2 can also be determined automatically by the application software 2, wherein, on the one hand, it can be provided that the application software 2 calculates equal proportions T1, in particular automatically when the availability value AN does not correspond to the zero value ZV, wherein the number of proportions T1 corresponds to the number of diagnostic devices 10; 10a comprised in the diagnostic system 1. In other words, the available credit can be used to evenly increase the analysis credits of all diagnostic devices 10; 10a. The second variable value N2 is then set for each diagnostic device 10; 10a according to an equal proportion T1. As an alternative to equal proportions T1, the application software can also calculate individual proportions T2. In this case, the diagnostic devices 10; 10a send usage data to the application software 2 in a method step S7, in particular at least data on the number of analyses performed and/or the usage frequency, wherein the application software 2 stores the usage data and creates individual usage profiles for the diagnostic devices 10; 10a based on this data. The usage profiles are used as the basis for calculating the individual proportions T2, such that the T2 proportions for diagnostic devices 10; 10a with a high usage frequency are set higher than the T2 proportions for diagnostic devices 10; 10a with a low usage frequency. The second variable value N2 is then determined for each diagnostic device 10; 10a in accordance with the proportion T2 calculated for the individual diagnostic device 10; 10a. In other words, the analysis credits are added to the available credits in accordance with the expected usage frequency of the respective diagnostic devices 10; 10a.

In a method step S6, the availability value AN is changed by performing the second operation depending on the second variable value N2, in particular reduced by subtracting the second variable value N2 from the availability value AN, and the second variable value N2 is transmitted to a diagnostic device 10a, or to a plurality of diagnostic devices 10a, in particular to the software module or modules 12. The internal value A1a is changed and stored depending on the transmitted second variable value N2 by continuing the second operation, in particular increased by adding the second variable value N2 to the internal value A1a. The new internal value A1a is transmitted to the user software 2. This results in an increase in the analysis credit from the availability value AN by an increase amount. The second operation and the associated change in the internal value and availability value can comprise one or more arbitrary operations executed by a processor in any number or value range, for example, addition of natural numbers, which can enable the functionality of an increase in the broadest sense. Advantageously, the second operation comprises two inverse operations, such as subtracting the second variable value N2 from the availability value AN and adding the second variable value N2 to the internal value A1, A1a.

When performing the method with a single diagnostic device 10, method step S6 is performed accordingly for the diagnostic device 10.

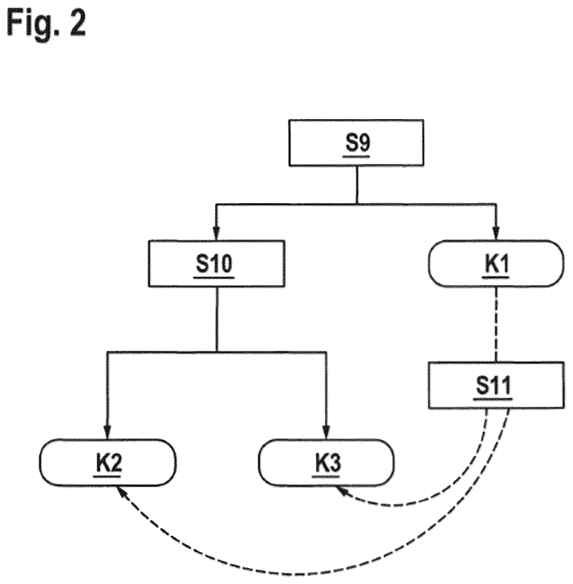

FIG. 2 shows a schematic illustration of how the diagnostic devices 10; 10a comprised in a diagnostic system 1 are assigned to categories K1; K2; K3. In a method step S9, the application software 2 checks the internal value A1; A1a of the diagnostic device or devices 10; 10a to determine whether the internal value A1; A1a has a value that does not correspond to the zero value ZV. If the internal value A1; A1a has a value that corresponds to the zero value ZV, the corresponding diagnostic device 10; 10a is assigned to a first category K1 by the application software 2.

The diagnostic devices 10; 10a which have not been assigned to the first category K1 are checked in a method step S10 by the application software 2 to determine whether the internal value A1; A1a at least corresponds to a value of a usage forecast for a defined period of time, in particular two weeks. The value of the usage forecast is generated by the application software 2 on the basis of individual usage data, for example the usage frequency, of the diagnostic devices 10; 10a. The usage forecast assumes a constant usage frequency for the defined period. In other words, it checks whether the corresponding diagnostic devices 10; 10a can be expected to operate for the defined period without the internal value A1; A1a and/or the analysis credit reaching the zero value ZV. If the internal value A1; A1a of a diagnostic device 10; 10a corresponds at least to the value of the usage forecast, the diagnostic device 10; 10a is assigned to a category K3 by the application software 2. If the internal value A1; A1a of a diagnostic device 10; 10a provides less analysis credit than is needed according to the usage forecast, the diagnostic device 10; 10a is assigned to a category K2 by the application software 2. If only a single diagnostic device 10 is comprised, the categorization into one of the categories K1; K2; K3 is performed for the single diagnostic device 10.

The categories K1, K2, and K3 are preferably highlighted in application software 2 by way of color coding, in particular in the form of a color traffic light, on a user interface.

A method step S11 may be provided, wherein the application software 2 determines a first variable value N1 and/or a second variable value N2 for a diagnostic device 10; 10a that has been classified into the first or second category K1; K2, which corresponds to a difference between the internal value A1; A1a of the diagnostic device 10; 10a and a limit value for classification into the second or third category K2; K3. In other words, the application software 2 can, in particular automatically or through user input, initiate a replenishment of analysis credits for a diagnostic device 10; 10a whose internal value A1; A1a cannot guarantee operation for a defined period of time. A reduction in the analysis credit balance of a diagnostic device 10; 10a may also be provided, in particular to make available credit balances for a top-up, wherein it is conceivable that the internal value A1; A1a is changed such that the limit value for a first or second category K1; K2 is exceeded or not exceeded. After changing the internal value A1; A1a according to the method of the disclosure, the categorization method is performed again, starting with method step S9.

The method steps explained in FIG. 1 and FIG. 2 are not limited to taking place in the sequence described. It is possible that the method steps may be performed in an alternative order and/or partially repeated and/or that further method steps may take place between the method steps described.

What is claimed is:

1. A method for performing analyses of biological samples using at least one diagnostic device in a diagnostic system, which comprises the diagnostic device and an external control unit connected via a communication connection, wherein the method enables control of the distribution of analysis credits between the diagnostic device and the external control unit in order to improve the operational readiness of the diagnostic system, the method comprising:
checking whether an internal value stored in a software module in a control device of the diagnostic device, which represents a number of analyses available on the device, corresponds to a zero value;
analyzing the biological sample by the diagnostic device if the internal value deviates from the zero value, in particular if it is greater than the zero value;
changing the internal value in the software module of the diagnostic device by an operation value of an analysis if the analysis of the biological sample is or has been performed;
performing a first operation performed by the processor of the control device or the external control unit in the presence of a communication connection with bi-directional data exchange between the diagnostic device and application software of the external control unit, whereby the internal value is changed, in particular reduced, as a function of a first variable value representing a number of analyses to be transmitted, and an availability value stored in the application software of the external control unit is changed, in particular increased, as a function of the first variable value, in particular increased; and/or
performing a second operation executed by the processor of the control device or the external control unit in the presence of a communication connection with bi-directional data exchange between the diagnostic device and the application software of the external control unit, whereby the available value stored in the application software of the external control unit is changed or reduced depending on a second variable value representing a number of analyses to be transmitted, in particular reduced, and the internal value is changed, in particular increased, depending on the second variable value.

2. The method according to claim 1, wherein:
if the first and/or second operation changes the internal value and/or the availability value to a value that is beyond the zero value based on the original internal value and/or availability value, in particular falls below or exceeds the zero value, the first and/or second operation is not executed and/or is aborted.

3. The method according to claim 1, wherein:
the application software checks whether a user input has been made at a user interface of the external control unit and sets the first and/or second variable value according to the user input.

4. The method according to claim 1, wherein:
the application software calculates a proportion of equal size for a plurality of diagnostic devices from the availability value automatically when the availability value reaches a value deviating from the zero value, and determines the second variable value according to the proportion.

5. The method according to claim 1, wherein:
usage data relating to the analyses performed, in particular by a plurality of diagnostic devices, preferably at least the number of analyses performed, is transmitted to the application software, wherein the application software stores the usage data and, on the basis of the usage data, creates and stores an individual usage profile for each of the diagnostic devices based on the usage data.

6. The method according to claim 5, wherein:
the application software calculates individual proportions from the availability value automatically when the availability value reaches a value deviating from the zero value, based on the usage profiles of a plurality of diagnostic devices, and determines the second variable value for the diagnostic devices according to the respective corresponding individual proportion.

7. The method according to claim 5, wherein:
the application software creates an individual usage forecast for a plurality of diagnostic devices based on the usage profiles, wherein the difference between an expected usage frequency, in particular for 14 days from the creation of the usage forecast, and the internal value is calculated.

8. The method according to claim 7, wherein:
a plurality of diagnostic devices are assigned to a respective category on the basis of the usage forecast, which category is highlighted in a graphical user interface of the application software.

9. The method according to claim 8, wherein:
the application software, when assigning a diagnostic device to a category, the first and/or second variable value is determined according to a difference between the internal value and a threshold value calculated on the basis of the usage forecast for assignment to another category.

10. A control device for a diagnostic device for performing analyses of biological samples in a diagnostic system, which is configured to perform the following method steps:
checking whether an internal value stored in a software module of the control device corresponds to a zero value in analyses when a biological sample is or was fed to the diagnostic device by way of a consumable material specific to the diagnostic device;
approving an analysis of the biological sample by the diagnostic device if the internal value deviates from the zero value, in particular if it is greater than the zero value;
changing the internal value in the software module of the diagnostic device by an operation value of an analysis if the analysis of the biological sample is being or has been performed;
performing a first operation with an existing communication connection with bi-directional data exchange between the control device and application software of an external control unit, whereby the internal value is changed, in particular reduced, as a function of a first variable value representative of a number of analyses, and an availability value stored in the application software of the external control unit is changed, in particular increased, as a function of the first variable value (N1); and/or
performing a second operation with an existing communication connection with bi-directional data exchange between the control device and the application software of an external control unit, whereby an availability value stored in the application software of the external control unit is changed, in particular reduced, as a function of a second variable value representative of a number of analyses, and the internal value is changed, in particular increased, as a function of the second variable value.

11. A diagnostic device for performing analyses of biological samples in a diagnostic system with the control device according to claim 10.

12. A computer program product comprising instructions which, when executed by a computer, cause the computer to perform a method for analyzing biological samples using at least one diagnostic device in a diagnostic system according to the method of claim 1.

13. The method according to claim 7, wherein:
a plurality of diagnostic devices are assigned to a respective category on the basis of the usage forecast, which category is highlighted in a graphical user interface of the application software visually.

14. The method according to claim 7, wherein:
a plurality of diagnostic devices are assigned to a respective category on the basis of the usage forecast, which category is highlighted in a graphical user interface of the application software visually as a color-coded traffic light.
